(19) United States
(12) Patent Application Publication
(10) Pub. No.: US 2011/0306801 A1
(43) Pub. Date: Dec. 15, 2011

(54) EXTRACTION AND SEPARATION PROCESSES FOR RECOVERY OF ORGANIC SOLUTES FROM FEED SOURCES AND APPARATUSES FOR PERFORMING SAME

(75) Inventor: Robert C. Schucker, The Woodlands, TX (US)

(73) Assignee: Trans Ionics Corporation, The Woodlands, TX (US)

(21) Appl. No.: 13/202,394

(22) PCT Filed: Feb. 19, 2010
(86) PCT No.: PCT/US10/24668

§ 371 Date: Aug. 19, 2011

Related U.S. Application Data
(60) Provisional application No. 61/153,852, filed on Feb. 19, 2009.
(60) Provisional application No. 61/248,702, filed on Oct. 5, 2009.

(51) Int. Cl. C07C 45/80; C07C 29/86; B01D 11/00; C07C 29/76

(52) U.S. Cl. 568/411; 568/917; 568/918; 422/261

(57) ABSTRACT

Methods for extracting an organic solute from a feed source using aromatic extraction solvents are described herein. Solvent modifiers such as, for example, cetyl alcohol may also be used to alter the properties of the solvent and improve the extraction. In some embodiments, the extraction solvent can be recycled after performing the extraction. In some embodiments, the organic solute may be separated from the extraction solvent after the extraction. For example, in some embodiments, the organic solute may be adsorbed on to 5 Å molecular sieve zeolites and then removed thereafter. Removal of the organic solute can take place by heating, applying a vacuum or a combination thereof. Apparatuses for extracting an organic solute from a feed source are also described herein.

### CROSS-REFERENCE TO RELATED APPLICATIONS

This application claims priority to U.S. Provisional Patent applications 61/153,852, filed Feb. 19, 2009, and 61/248,702, filed Oct. 5, 2009, each of which is incorporated by reference herein in its entirety.

### STATEMENT REGARDING FEDERALLY SPONSORED RESEARCH

Not applicable.

### BACKGROUND

Alcohol-based fuels such as, for example, ethanol and butanol, as well as chemicals such as, for example, acetone, may be produced from corn or sugar. Such fuels and chemicals may also be produced from cellulosic feeds such as, for example, switch grass, corn stover, bagasse, tree bark and sawdust, through fermentation or other biochemical processes. Fermentation of corn and sugar typically produces fermentation broths containing ethanol at concentrations above about 15 percent by volume in an aqueous medium. In contrast, fermentation of cellulosic feedstocks typically produces a range of chemical products that are each usually present at a concentration of less than about 5 percent by volume, and often less than 2 percent by volume.

Traditionally, distillation has been used to recover these products from the aqueous medium. More dilute feedstocks generally require larger distillation systems and consume considerably more energy than when more concentrated fermentation feedstocks are separated. Further, since both ethanol and butanol distill azeotropically with water, both capital cost and energy consumption for their recovery are higher.

While significant work has been performed over the past several decades on ways to better convert various cellulosic and non-cellulosic feedstocks into valuable chemicals and biofuels, considerably fewer studies have been performed on ways to reduce the cost and improve the efficiency of recovering these chemicals from the resulting fermentation broths. Among the techniques proposed for improved separation include stripping, adsorption, liquid-liquid extraction, pervaporation and membrane solvent extraction. However, none of the proposed techniques have resulted in a significant improvement over distillation in terms of cost and efficiency. Many of the aforementioned processes are driven by the presence of a concentration gradient which makes them relatively unsuitable when low concentration fermentation broths are treated. Further, some of the techniques often result in emulsification when applied to fermentation broths, which also makes them energy inefficient.

In particular regard to liquid-liquid extraction, there is a further inherent inefficiency in that a finite amount of residual extraction solvent typically remains dissolved in the raffinate phase after extraction. Typically, a distillation is used to recover the residual extraction solvent, and a second distillation is often used to remove extraction solvent from an extracted solute in an extract phase. These secondary distillations add to the cost and energy inefficiency of current liquid-liquid extraction processes, particularly when the concentration of extracted solute is relatively small.

In view of the foregoing, there is a need for improved extraction processes and apparatuses that can reduce energy used to recover an extracted solute, particularly when more dilute feed sources are being processed. In order for production of ethanol, butanol and other chemical compounds from biological feedstocks to be economically viable, there is a need more energy efficient processes to achieve their separation and recovery.

### SUMMARY

In various embodiments, the present disclosure describes methods for extracting an organic solute from a feed source. The method includes providing a feed source, transferring the feed source to a liquid-liquid extraction unit containing a plurality of equilibrium stages; contacting the feed source with an extraction solvent in each of the plurality of equilibrium stages, and recovering a raffinate phase substantially depleted of the organic solute and an extract phase substantially enriched in the organic solute. The feed source contains the organic solute. The extraction solvent includes at least one aromatic solvent and is substantially immiscible with the feed source. Contacting includes transferring at least a portion of the organic solute from the feed source to the extraction solvent. The organic solute is dissolved in the extraction solvent in the extract phase. In some embodiments, the methods further include passing the extract phase through at least one bed of 3 Å molecular sieve zeolites and then passing the extract phase through at least one bed of 5 Å molecular sieve zeolites, which adsorb the organic solute. In some embodiments, the methods further include recovering the organic solute from the 5 Å molecular sieve zeolites.

In other various embodiments of methods for extracting an organic solute from a feed source include providing a feed source, transferring the feed source to a liquid-liquid extraction unit containing a plurality of equilibrium stages; contacting the feed source with an extraction solvent in each of the plurality of equilibrium stages, and recovering a raffinate phase substantially depleted of the organic solute and an extract phase substantially enriched in the organic solute. The feed source contains the organic solute, which may be, for example, acetone, butanol, isobutanol, ethanol, or various combinations thereof. The extraction solvent includes cetyl alcohol and at least one aromatic solvent and is substantially immiscible with the feed source. Contacting includes transferring at least a portion of the organic solute from the feed source to the extraction solvent. The organic solute is dissolved in the extraction solvent in the extract phase. In some embodiments, the methods further include passing the extract phase through at least one bed of 3 Å molecular sieve zeolites and then passing the extract phase through at least one bed of 5 Å molecular sieve zeolites, which adsorb the organic solute. In some embodiments, the methods further include recovering the organic solute from the 5 Å molecular sieve zeolites.

In some embodiments, the present disclosure describes methods for extracting an organic solute from water. The methods include providing an organic solute dissolved in water and extracting the organic solute from the water using an extraction solvent containing cetyl alcohol.

In some embodiments, methods for separating an organic solute from a feed source include providing a feed source, passing the feed source through at least one bed of 3 Å molecular sieve zeolites to form a dewatered feed source, and then passing the dewatered feed source through at least one bed of 5 Å molecular sieve zeolites to adsorb the organic solute and form a substantially solute-free dewatered feed source. The substantially solute-free dewatered feed source includes spent extraction solvent. In some embodiments, the methods further include recovering the organic solute from the 5 Å molecular sieve zeolites.

In other various embodiments, the present disclosure describes apparatuses containing a solvent input line, a solvent transfer line, a first bed of 3 Å molecular sieve zeolites and a second bed of 3 Å molecular sieve zeolites linked to the solvent input line, a first bed of 5 Å molecular sieve zeolites and a second bed of 5 Å molecular sieve zeolites linked to the first bed 3 Å molecular sieve zeolites and the second bed of 3 Å molecular sieve zeolites by the solvent transfer line, and an output line linked to the first bed of 5 Å molecular sieve zeolites and the second bed of 5 Å molecular sieve zeolites. In some embodiments, either or both of the beds of the 3 Å molecular sieve zeolites or the beds of the 5 Å molecular sieve zeolites are operable in a swing bed fashion.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 shows a schematic of an illustrative liquid-liquid extraction unit;

FIG. 2 shows a schematic of an illustrative liquid-liquid extraction unit in which an electric field is applied to the extraction vessel;

FIG. 3 shows a schematic of an illustrative liquid-liquid extraction unit in which multiple extraction vessels are operated in parallel;

FIG. 4 shows an illustrative schematic of an extraction solvent treatment system;

FIG. 5 shows an illustrative distribution coefficient plot for the extraction of ethanol from water using mixed xylenes;

FIG. 6 shows an illustrative plot of ethanol content as a function of contact time with 5 Å zeolite pellets; and

FIG. 7 shows an illustrative plot of ethanol weight fraction versus volume of effluent collected.

### DETAILED DESCRIPTION

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto. Drawings are not necessarily to scale.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009. Definitions and/or interpretations should not be incorporated from other patent applications, patents, or publications, related or not, unless specifically stated in this specification or if the incorporation is necessary for maintaining validity.

Most previous work on liquid-liquid extraction of dilute feedstocks has centered upon in-situ rather than ex-situ recovery of chemical products produced from processes such as, for example, fermentation. As a result, solvent choices have generally been limited to those that are non-toxic to bacteria and extraction temperatures have been limited to those at or below which fermentation bacteria can survive, typically about 40° C. Such conditions are not optimal for extraction and often lead to poor mass transfer and emulsion formation.

In various embodiments, the present disclosure describes methods for extracting an organic solute from a feed source. The method includes providing a feed source, transferring the feed source to a liquid-liquid extraction unit containing a plurality of equilibrium stages; contacting the feed source with an extraction solvent in each of the plurality of equilibrium stages, and recovering a raffinate phase substantially depleted of the organic solute and an extract phase substantially enriched in the organic solute. The feed source contains the organic solute. The extraction solvent includes at least one aromatic solvent and is substantially immiscible with the feed source. Contacting includes transferring at least a portion of the organic solute from the feed source to the extraction solvent. The organic solute is dissolved in the extraction solvent in the extract phase. In some embodiments, the methods further include passing the extract phase through at least one bed of 3 Å molecular sieve zeolites and then passing the extract phase through at least one bed of 5 Å molecular sieve zeolites, which adsorb the organic solute. In some embodiments, the 3 Å molecular sieve zeolites are optional. In some embodiments, the methods further include recovering the organic solute from the 5 Å molecular sieve zeolites.

Other various embodiments of methods for extracting an organic solute from a feed source include providing a feed source, transferring the feed source to a liquid-liquid extraction unit containing a plurality of equilibrium stages; contacting the feed source with an extraction solvent in each of the plurality of equilibrium stages, and recovering a raffinate phase substantially depleted of the organic solute and an extract phase substantially enriched in the organic solute. The feed source contains the organic solute, which may be, for example, acetone, butanol, isobutanol, ethanol, or various combinations thereof. The extraction solvent includes cetyl alcohol and at least one aromatic solvent and is substantially immiscible with the feed source. Contacting includes transferring at least a portion of the organic solute from the feed source to the extraction solvent. The organic solute is dissolved in the extraction solvent in the extract phase. In some embodiments, the methods further include passing the extract phase through at least one bed of 3 Å molecular sieve zeolites and then passing the extract phase through at least one bed of 5 Å molecular sieve zeolites, which adsorb the organic solute. In some embodiments, the 3 Å molecular sieve zeolites are optional. In some embodiments, the methods further include recovering the organic solute from the 5 Å molecular sieve zeolites.

In various embodiments, feed sources of the present disclosure are fermentation feed sources. Accordingly, such fermentation feed sources are typically dilute aqueous feed sources. In some embodiments, the feed source includes a biofuel (e.g., acetone, butanol, ethanol, propanol, isopropanol, and/or isobutanol). In some embodiments, the feed source is substantially devoid of solids. In some embodiments, the feed source may be filtered or decanted prior to having the organic solute removed by extraction.

Methods of the present disclosure are advantageous over the aforementioned attempted improvements in liquid-liquid extraction because they are ex situ rather than in situ in nature. Accordingly, the methods advantageously permit aromatic solvents alone or in combination with solvent modifiers to be used, since many aromatic solvents are toxic to fermentation bacteria. Furthermore, methods of the present disclosure are advantageous in that there is no particular limit on the extraction temperature, other than extraction solvent boiling point, since there is no concern about bacterial viability. Similarly, there is no description in past work demonstrating the use of an extraction solvent or extraction solvent component that is solid at room temperature but melts at temperature above about 50° C.

Aromatic extraction solvents of the present disclosure include, for example, benzene, toluene, xylenes (o, m, or p-isomers), and combinations thereof. In various embodiments, the extraction solvents of the present disclosure have kinetic diameters greater than about 6 Å. Aromatic extraction solvents have excellent affinity for alcohols versus water, yet their use in liquid-liquid extraction for biofuel separation is presently unknown. The aromatic extraction solvent has a boiling point in excess of about 100° C. in some embodiments, in excess of about 150° C. in other embodiments, and in excess of about 200° C. in yet additional embodiments.

In some embodiments, extraction solvents of the present disclosure further include a solvent modifier. Such solvent modifiers can change the polarity or other property of the extraction solvent and alter the affinity of the solvent for extracting a particular organic solute or class of solute. In some embodiments, solvent modifiers may be, for example, long chain alcohols, long chain fatty acids, esters of long chain fatty acids, naturally-occurring oils and various combinations thereof. As used herein, long chain alcohols (e.g., 1-dodecanol, cetyl alcohol, and stearyl alcohol), fatty acids (e.g., oleic acid, linoleic acid, linolenic acid, stearic acid), and esters of long chain fatty acids (e.g., methyl oleate and biodiesel) refer to molecules having carbon chain lengths greater than about 10 carbons. Naturally-occurring oils and fats include such substances as, for example, corn oil, soybean oil, olive oil, castor oil and various combinations thereof. In some embodiments, the solvent modifier may be a solid at room temperature. In some embodiments, the solvent modifier includes cetyl alcohol.

The amount of the solvent modifier may range between about 0.1 weight percent and about 99.9 weight percent of the extraction solvent in some embodiments, between about 5 weight percent and about 90 weight percent in other embodiments, and between about 25 weight percent and about 75 weight percent in still other embodiments. The primary aromatic solvent(s) form the remainder of the extraction solvent.

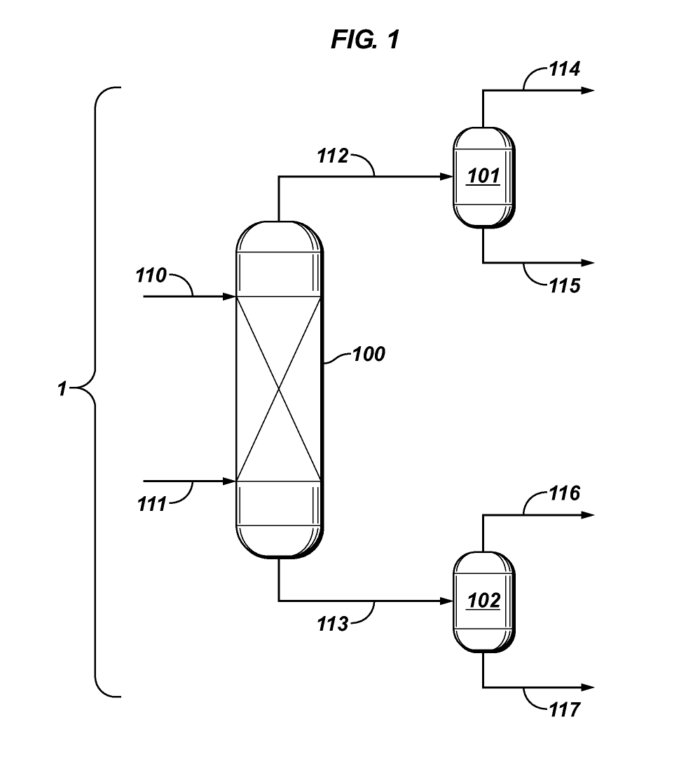

FIG. 1 shows a schematic of an illustrative liquid-liquid extraction unit 1 suitable for extracting organic solutes from feed sources of the present disclosure. In an embodiment, the feed source is a dilute solution of organic solutes in water in which the concentration of each organic solute is less than about 3 percent by volume and the total concentration of organic solutes is less than about 6 percent by volume. However, one of ordinary skill in the art will recognize that the methods of the present disclosure may be practiced using feed sources that are either more or less concentrated than that noted above. The temperature of the feed source as produced from fermentation is generally in a range from about 25° C. to about 40° C. A heat exchanger (not shown) may be used to adjust the feed source temperature prior to performing liquid-liquid extraction in order to achieve the optimal temperature for separation.

Referring to FIG. 1, the feed source enters the top of liquid-liquid extraction unit 1 through line 110 and is dispersed into a continuous phase and moves downward through the column contained in extraction vessel 100. An extraction solvent enters the bottom of liquid-liquid extraction unit 1 through line 111 and moves upward through the column contained in extraction vessel 100. The extract phase substantially enriched in organic solute exits extraction vessel 100 through line 112. The raffinate phase substantially depleted in organic solute phase exits extraction vessel 100 through line 113. In some embodiments, extraction vessel 100 contains means for enhancing the contacting of the two phases (e.g., sieve trays, baffles, packing or agitators) as well as disengaging zones at the top and bottom of the column, wherein the phases separate effectively to allow for efficient recovery.

In liquid-liquid extraction, it is known that a small amount of residual extraction solvent may be dissolved in the raffinate phase. In some embodiments, methods of the present disclosure further include removing any residual extraction solvent from the raffinate phase. Referring again to FIG. 1, raffinate phase in line 113 may be transferred into separator vessel 102, which removes the residual extraction solvent to form a stream in line 116 substantially enriched in extraction solvent and a stream in line 117 substantially depleted in extraction solvent. In some embodiments, the extraction solvent may be subsequently recycled. Separator vessel 102 may be a distillation column or any other separation apparatus that is capable of separating the extraction solvent from the raffinate phase. In some embodiments, the separation apparatus may function via adsorption of the extraction solvent.

In some embodiments, methods of the present disclosure further include removing the organic solute from the extract phase and recycling the extraction solvent after removing the organic solute. Referring still to FIG. 1, the extract phase in line 112 is fed to a second separator vessel 101, which produces a product stream in line 114 containing the organic solute and a solvent stream in line 115 substantially depleted in the organic solute and containing the extraction solvent. In various embodiments, separator vessel 101 may be a distillation column or a separation apparatus functioning by adsorption.

One of ordinary skill in the art will recognize that many different extraction solvents and combinations of extraction solvents and solvent modifiers may be used to extract an organic solute from dilute feed streams and that many different contactor types and flow configurations may be used to carry out the extraction process.

Operating temperature of the liquid-liquid extraction unit is a variable for the extraction process. The operating temperature is between about 25° C. and about 100° C. in some embodiments, between about 50° C. and about 90° C. in other embodiments, and between about 75° C. and about 80° C. in still other embodiments.

Operating pressure of the liquid-liquid extraction unit is not particularly critical. In general the operating pressure is only kept high enough to maintain all components in the liquid phase at the operating temperature. In some embodiments, separator vessel 101 may be a vacuum flash drum in which the pressure may range from about 1 ton (1 torr=1 mm Hg) to about 750 torr, or from about 5 torr to about 500 torr or from about 10 torr to about 100 torr.

Several advantages are realized through the above described extraction procedures. Namely, the use of a standard solvent extractor configuration simplifies the commercial application of this process, since such solvent extractors are in common use in petrochemical plants and refineries. Further, solvent extractors are substantially cheaper than distillation apparatuses because they need not be made of stainless steel. In some embodiments of the present disclosure, a packed column arrangement may be used because of its inherent simplicity.

In some embodiments, methods of the present disclosure further include applying an electric field to the extraction solvent and the feed source while contacting occurs. The electric field increases a surface area of the extraction solvent dispersed in the feed source. Use of an electric field to enhance mass transfer during extraction is described in U.S. Pat. Nos. 4,767,515 and 5,385,658, each of which are incorporated herein by reference. The electric field produces fine droplets of the extraction solvent dispersed within a continuous phase (i.e., the feed source). Initial extraction solvent droplets are shattered by the high intensity electric field, which subsequently recombine to form smaller droplets than the initial extraction solvent droplets and have a greater combined surface area than those initially present. In some embodiments, the electric field is a pulsed electric field. Application of an electric field advantageously increases the number of theoretical stages in a multi-stage liquid-liquid extraction unit.

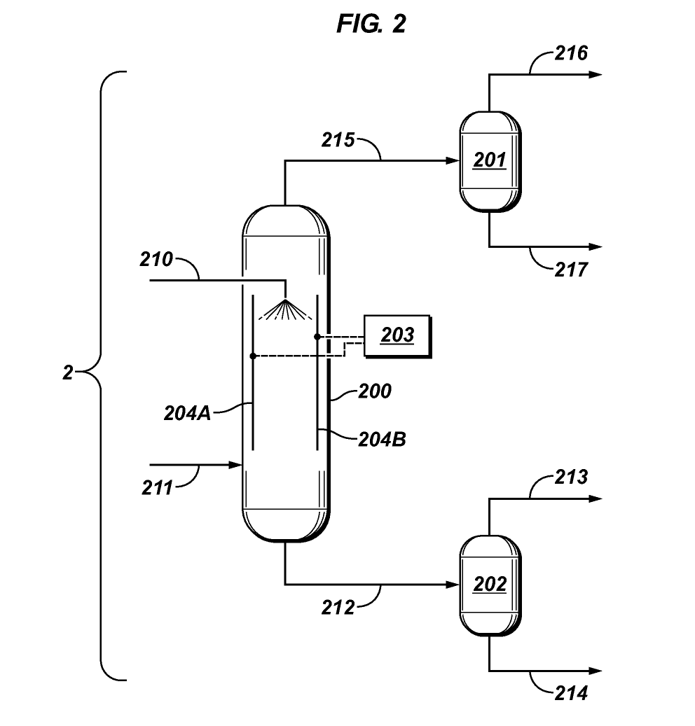

FIG. 2 shows a schematic of an illustrative liquid-liquid extraction unit 2 in which an electric field is applied to the extraction vessel. Illustrative parameters are the same as those set forth above for FIG. 1. As in FIG. 1, the feed source enters liquid-liquid extraction unit 2 through line 210 and moves through the column contained in extraction vessel 200. Extraction solvent enters liquid-liquid extraction unit 2 through line 211 and moves upward through the column contained in extraction vessel 200. The extract phase exits through line 215. The raffinate phase exits through line 212. Extraction vessel 200 contains a means for producing a pulsed electric field, such as described hereinabove. In the embodiment shown in FIG. 2, droplets of a dispersed aqueous phase are introduced into a countercurrent flow of a continuous phase (e.g., feed stream). Droplets of the dispersed aqueous phase have a first surface area and are allowed to free fall through the continuous phase. Introduction of the dispersed aqueous phase is made between two electrodes, designated 204A and 204B, which apply a high intensity pulsed electric field to the droplets of the dispersed phase. The electric field shatters the droplets of dispersed phase into many smaller droplets, which form an emulsion of smaller droplets in the continuous phase. The smaller droplets have a combined total surface area that is greater than that of the total surface area of the original droplets. The smaller droplets subsequently coalesce to reform larger droplets, which are stable in the electric field. The electric field may be provided by a high voltage, low amperage A/C source (i.e. ˜20 kV, 60 Hz A/C system) or a pulsed D/C system generated by electronic controller 203. The pulse rate of the pulsed electric field may be 20-60 Hz or 60-120 Hz such that each droplet has a natural oscillation frequency and the pulsed frequency applied is in the vicinity of the natural oscillation frequency.

Referring again to FIG. 2, raffinate phase in line 212 may be transferred into separator vessel 202, which removes the residual extraction solvent to form a stream in line 213 substantially enriched in extraction solvent and a stream in line 214 substantially depleted in extraction solvent. In some embodiments, the extraction solvent may be subsequently recycled. Separator vessel 202 may be a distillation column or any other separation apparatus that is capable of separating the extraction solvent from the raffinate phase. In some embodiments, the separation apparatus may function via adsorption of the extraction solvent.

Referring still to FIG. 2, the extract phase in line 215 is fed to a second separator vessel 201, which produces a product stream 216 containing the organic solute and a solvent stream 217 substantially depleted in the organic solute and containing the extraction solvent. In various embodiments, separator vessel 201 may be a distillation column or a separation apparatus functioning by adsorption.

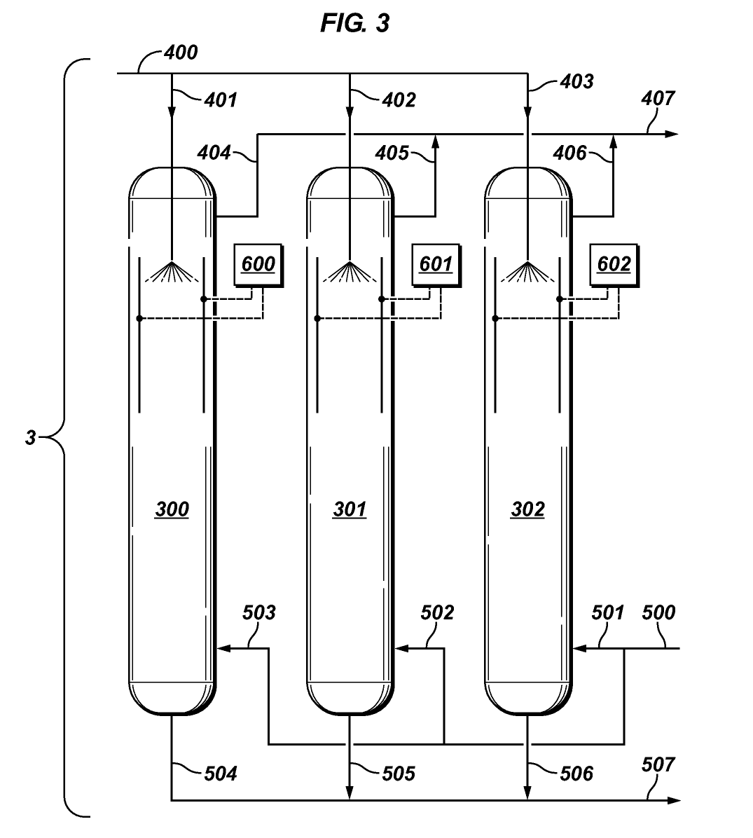

FIG. 3 shows a schematic of an illustrative liquid-liquid extraction unit 3 in which multiple extraction vessels are operated in parallel. As in the embodiment shown in FIG. 2, each of the extraction units contains a means for producing a pulsed electric field. In the embodiment shown in FIG. 3, feed stream entering through line 400 is divided into approximately equal parts, each of which is fed to the individual extraction vessels 300, 301 and 302 through lines 401, 402 and 403. Extraction solvent enters the extraction unit 3 through line 500 before being diverted into the individual extraction vessels 300, 301 and 302 through lines 501, 502 and 503. In some embodiments, extraction vessels 300, 301 and 302 are arranged such that the flow to each vessel does not exceed a fixed maximum flow rate and thus ensures good fluid-fluid contacting. Raffinate phase exits through lines 504, 505 and 506 and is collected in manifold 507. Extract phase exits through lines 404, 405 and 406 and collects in manifold 407. Pulsed electric fields are produced in each extraction unit and regulated by controllers 600, 601 and 602. Further treatment of the extract phase in manifold 407 and the raffinate phase in manifold 507 is performed comparably to that described hereinabove for FIG. 2.

In some embodiments, methods of the present disclosure further include passing the extract phase through at least one bed of 3 Å molecular sieve zeolites and then passing the extract phase through at least one bed of 5 Å molecular sieve zeolites. In some embodiments, treatment with the 3 Å molecular sieve zeolites is optional. The 5 Å molecular sieve zeolites adsorb the organic solute from the extract phase. In some embodiments, methods of the present disclosure further include recovering the organic solute from the 5 Å molecular sieve zeolites.

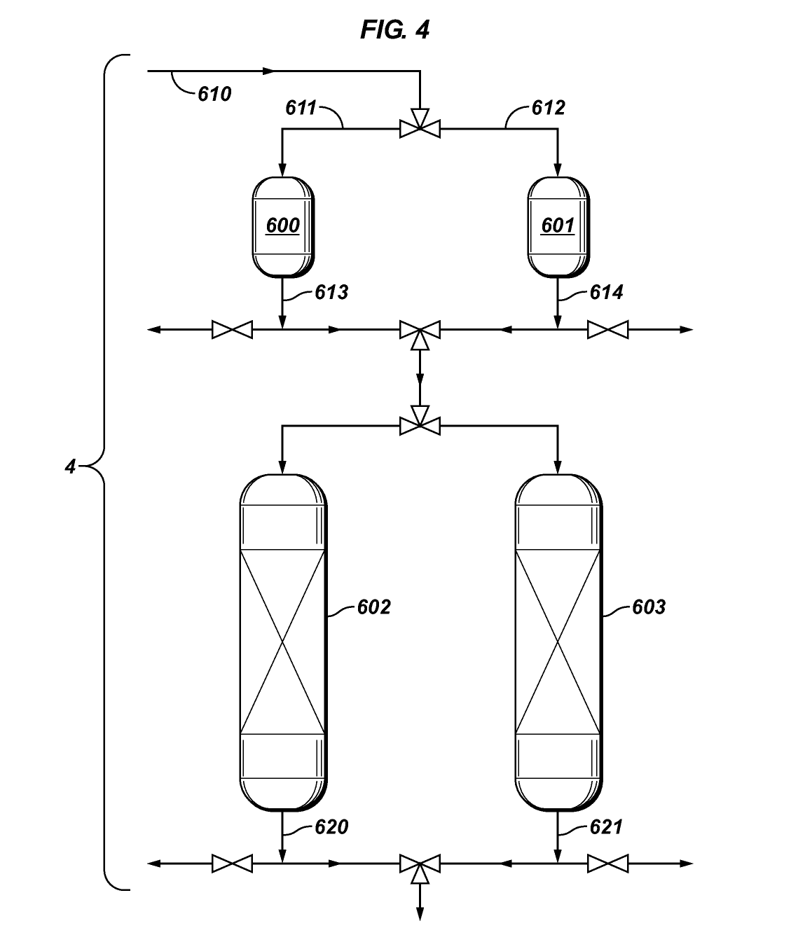

The extract phase produced as described hereinabove may be further processed according to the embodiment shown in FIG. 4 in order to separate the organic solute from the extraction solvent. FIG. 4 shows an illustrative schematic of an extraction solvent treatment system 4. The extraction solvent treatment system is coupled to a liquid-liquid extraction unit (not shown).

As shown in FIG. 4, extract phase from the liquid-liquid extraction unit in line 610 (equivalent to line 112 in FIG. 1 or line 215 in FIG. 2) contains a mixture of organic solute (e.g, ethanol and/or butanol) and extraction solvent with some residual water content. Embodiments of the present disclosure allow this residual water content to be removed and form a dewatered extract phase. As shown in the embodiment of FIG. 4, extract phase in line 610 is split into lines 611 and 612, which are connected to beds of activated 3 Å molecular sieve zeolites 600 and 601. Beds 600 and 601 are capable of being operated in a typical swing bed fashion. That is, in some embodiments, if bed 600 is adsorbing water from the extract phase, then bed 601 is being regenerated. When bed 600 is spent, it can then be regenerated while bed 601 is being used to adsorb water from the extract phase. Such swing bed operation advantageously allows continuous operation of the system. Alternately, beds 600 and 601 may be operated in parallel in some embodiments, rather than in a swing bed fashion.

Beds 600 and 601 allow the extract phase to be dewatered before the organic solute is removed from the extract phase. Extract phase passing through beds 600 and 601 interacts with the activated 3 Å molecular sieve zeolites contained therein which, because of their small pore size, selectively adsorb water from the extract phase. Dewatered extract phase leaving beds 600 and 601 in lines 613 and 614 is essentially water-free but still contains organic solute and extraction solvent. This dewatered extract phase is then passed to beds of activated 5 Å molecular sieve zeolites 602 and 603. As with the beds of activated 3 Å molecular sieve zeolites, the beds of activated 5 Å molecular sieve zeolites may be operated in a swing bed fashion in some embodiments and in parallel in other embodiments. The 5 Å molecular sieve zeolites have pores that are large enough to accept ethanol, butanol and other small molecule biofuels (e.g., acetone) but not extraction solvent molecules having a molecular diameter of >4 Å. Therefore, dewatered extract phase entering beds 602 and 603 has the organic solute selectively adsorbed by the 5 Å molecular sieve zeolites, and dewatered, solute-free extract phase then exits beds 602 and 603 through lines 620 and 621. Dewatered solute-free extract phase, which is essentially pure extraction solvent, can then be recycled to the liquid-liquid extraction system or otherwise reused. The process is performed isothermally thereby significantly reducing energy consumption of relative to distillation.

Organic solute (e.g., ethanol or butanol) adsorbed on the 5 Å molecular sieve zeolites can be recovered when beds 602 and 603 are regenerated. In an embodiment, either bed 602 or 603 (when operated in a swing bed fashion) is drained of all liquid surrounding the 5 Å molecular sieve zeolites and then heated (e.g., passing a hot gas over the bed), placed under vacuum, or both. In some embodiments, heating can also be easily accomplished through coils built into the bed. Vaporized organic solute can be withdrawn from the bed as it is desorbed from the 5 Å molecular sieve zeolites. The vaporized organic solute typically consists primarily of the organic solute and a minor amount of residual extraction solvent not removed from the bed. The vaporized organic solute can be passed through condensers (not shown) which remove most of the residual extraction solvent. Any residual extraction solvent not removed is typically acceptable as a denaturant in ethanol systems. Alternately, adsorbed organic solute can be removed from beds 602 and 603 simultaneously, but such parallel operation results in interruption of the continuous extraction process. Operation in a swing-bed fashion advantageously permits continuous operation of the system.

The arrangement of vessels and the use of control valves to guide streams where desired in a system containing beds operated in a swing bed fashion is well known to those of ordinary skill in the art. The number and orientation of valves and beds shown in the embodiments described herein should be considered illustrative, and other variations not explicitly drawn herein lie within the spirit and scope of the present disclosure.

In some embodiments, methods for separating an organic solute from a feed source include providing a feed source, passing the feed source through at least one bed of 3 Å molecular sieve zeolites to form a dewatered feed source, and then passing the dewatered feed source through at least one bed of 5 Å molecular sieve zeolites to adsorb the organic solute and form a substantially solute-free dewatered feed source. The substantially solute-free dewatered feed source includes spent extraction solvent. In some embodiments, the 3 Å molecular sieve zeolites are optional.

In some embodiments, the methods further include recovering the organic solute from the 5 Å molecular sieve zeolites. In some embodiments, recovering the organic solute is performed during regeneration of the 5 Å molecular sieve zeolites. In some embodiments, recovering takes place by heating the 5 Å molecular sieve zeolites. In other embodiments, recovering takes place by applying a vacuum to the 5 Å molecular sieve zeolites. In still other embodiments, recovering takes place by heating and applying a vacuum to the 5 Å molecular sieve zeolites. As noted above, application of heat, vacuum, or a combination thereof results in desorption of the adsorbed organic solutes from the 5 Å molecular sieve zeolites, resulting in their removal as a vapor. Once the organic solutes have been desorbed from the 5 Å molecular sieve zeolites, the bed containing the zeolites is regenerated and ready to adsorb additional organic solutes of an appropriate molecular diameter.

In some embodiments of the methods, there are two beds of 3 Å molecular sieve zeolites and two beds of 5 Å molecular sieve zeolites. In some embodiments, the two beds of 3 Å molecular sieve zeolites and the two beds of 5 Å molecular sieve zeolites are operated in a swing bed fashion. That is, when one bed is being regenerated and the adsorbed organic solutes are being removed, the other bed is actively being used to adsorb organic solutes from the feed source. However, in other embodiments, the two beds of 3 Å molecular sieve zeolites and the two beds of 5 Å molecular sieve zeolites are operated in parallel.

In some embodiments of the methods, the spent extraction solvent is recycled. For example, in some embodiments, the spent extraction solvent may be returned to a liquid-liquid extraction unit in order to perform removal of additional organic solute from a feed source. In some embodiments, the spent extraction solvent may be further purified before being recycled, if desired.

In some embodiments of the methods described herein, the organic solute has a molecular diameter of not more than about 4 Å. Such molecular diameters allow the organic solute molecules to be selectively adsorbed by 5 Å molecular sieve zeolites. Furthermore, aromatic extraction solvents and solvent modifiers such as, for example, cetyl alcohol are too large to be adsorbed and retained by the 5 Å molecular sieve zeolites. In some embodiments of the methods, the organic solute is an alcohol. In some embodiments, the alcohol is ethanol. In other embodiments of the methods, the alcohol is butanol.

In some embodiments of the methods, the extraction solvent includes at least one aromatic solvent. In some embodiments, the extraction solvent further includes a solvent modifier such as, for example, cetyl alcohol.

In other various embodiments, the present disclosure describes apparatuses containing a solvent input line, a solvent transfer line, a first bed of 3 Å molecular sieve zeolites and a second bed of 3 Å molecular sieve zeolites linked to the solvent input line, a first bed of 5 Å molecular sieve zeolites and a second bed of 5 Å molecular sieve zeolites linked to the first bed 3 Å molecular sieve zeolites and the second bed of 3 Å molecular sieve zeolites by the solvent transfer line, and an output line linked to the first bed of 5 Å molecular sieve zeolites and the second bed of 5 Å molecular sieve zeolites. In some embodiments, either or both of the beds of the 3 Å molecular sieve zeolites or the beds of the 5 Å molecular sieve zeolites are operable in a swing bed fashion. In some embodiments, the apparatuses further include a heater coupled to the first bed of 5 Å molecular sieve zeolites and the second bed of 5 Å molecular sieve zeolites.

In some embodiments, the present disclosure describes methods for extracting an organic solute from water. The methods include providing an organic solute dissolved in water and extracting the organic solute from water using an extraction solvent containing cetyl alcohol. In some embodiments, extracting takes place in a liquid-liquid extraction unit. In some embodiments, the liquid-liquid extraction unit contains a plurality of equilibrium stages. In some embodiments, the organic solute is an alcohol.

### EXAMPLES

The following examples are provided to more fully illustrate some of the embodiments disclosed hereinabove. It should be appreciated by those of ordinary skill in the art that the techniques disclosed in the examples that follow represents techniques that constitute illustrative modes for practice of the disclosure. Those of ordinary skill in the art should, in light of the present disclosure, appreciate that many changes can be made in the specific embodiments that are disclosed and still obtain a like or similar result without departing from the spirit and scope of the disclosure.

### Example 1

Calculation of Distribution Coefficients and Extraction of Simulated Feed Source Solutions. One measure of the effectiveness of a solvent for extracting solutes is termed the distribution coefficient, which is calculated as follows in Formula (1):

DC=(Wₒ/Wₛ)ₑₓₜ/(Wₒ/W_{w})_{raff} (1)

where:

### DC=the Distribution Coefficient

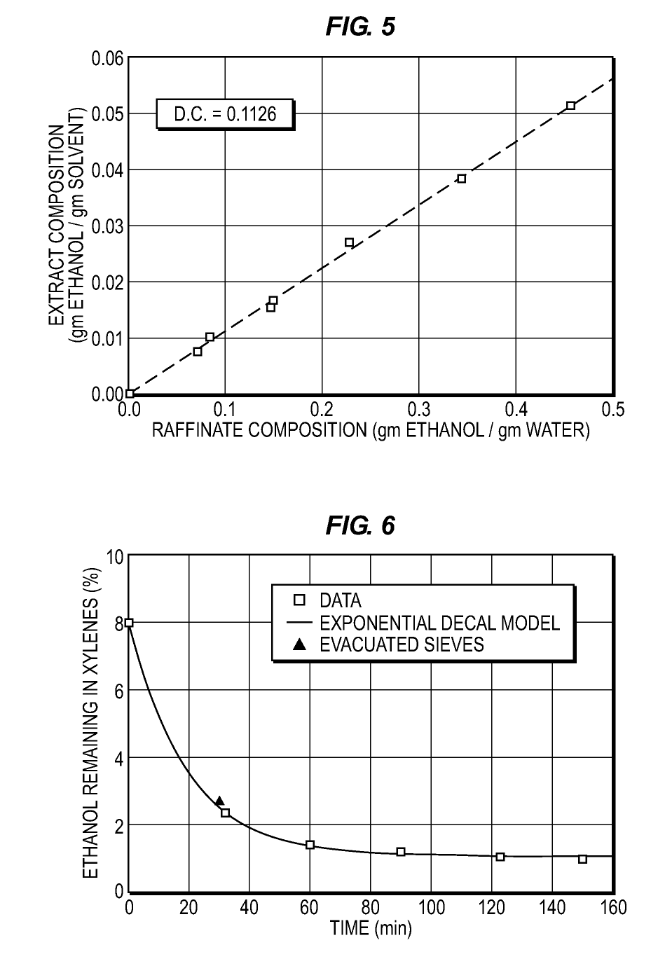

Wₒ=weight of organic solute (gm)
Wₛ=weight of solvent (gm)
W_{w}=weight of water (gm)
ext=indicates extract phase
raff=indicates raffinate phase
The distribution coefficient is typically determined by carrying out a number of extractions on feeds sources having a range of organic solute concentrations (at a constant solvent to feed source ratio), analyzing extract and raffinate samples and plotting [Wₒ/Wₛ]ₑₓₜ versus [Wₒ/W_{w}]_{raff}. The slope of this line is the distribution coefficient. FIG. 5 shows an illustrative distribution coefficient plot for the extraction of ethanol from water using mixed xylenes.

Feed solutions of varying concentrations were prepared by mixing known amounts of pure, anhydrous ethanol (or butanol or ethanol plus butanol plus acetone) and deionized water. Two milliliters of each feed were then placed in a 15 milliliter glass centrifuge tube equipped with a plastic screw-on cap (having a small round hole in its center) and silicone rubber septum. Solvent was added at the desired solvent to feed ratio, and the centrifuge tubes were equilibrated at several temperatures with frequent agitation. Samples of both phases were then withdrawn using a syringe and analyzed by gas chromatography. Results were plotted in a similar manner to that described above and shown in FIG. 5. The distribution coefficient was determined from the slope of the resulting line by linear regression of the data.

### Example 2

Aromatic Extraction Solvents For Extraction of Ethanol. Mixed xylenes (a mixture of o-xylene, m-xylene and p-xylene), which is a common hydrocarbon solvent, was tested as an extraction solvent for ethanol-containing feed streams at a solvent:feed ratio of 5:1 at 40° C., 60° C. and 75° C. Equilibrium data and calculated distribution coefficients are shown in TABLE 1 below.

TABLE 1

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.10 | 0.0699 | 0.0076 | 0.1126 | 0.998 |
| | 0.20 | 0.1457 | 0.0154 | | |
| | 0.40 | 0.3429 | 0.0383 | | |
| | 0.10 | 0.0701 | 0.0075 | | |
| | 0.20 | 0.1479 | 0.0166 | | |
| | 0.30 | 0.2267 | 0.0270 | | |
| | 0.50 | 0.4566 | 0.0512 | | |
| | 0.12 | 0.0826 | 0.0101 | | |
| 60 | 0.10 | 0.0788 | 0.0063 | 0.0815 | 0.999 |
| | 0.30 | 0.2695 | 0.0227 | | |
| | 0.50 | 0.5463 | 0.0442 | | |
| 40 | 0.10 | 0.0903 | 0.0042 | 0.0592 | 0.994 |
| | 0.10 | 0.0892 | 0.0041 | | |
| | 0.30 | 0.3102 | 0.0173 | | |
| | 0.50 | 0.6094 | 0.0370 | | |

As shown in Table 1, mixed xylenes gave a good distribution coefficient for ethanol that increased with increasing operating temperature. Further, use of mixed xylenes produced no emulsions and resulted in rapid separation from the aqueous feed solutions due to the large difference in density between the solvent and the feed solution.

### Example 3

Cetyl Alcohol as an Extraction Solvent For Ethanol. Cetyl alcohol was used as a solvent for ethanol extraction. Because cetyl alcohol melts between 47° C. and 50° C. and therefore is a solid at both room temperature (25° C.) and typical fermentation temperatures (40° C.), it has been overlooked as an extraction solvent. Cetyl alcohol was tested at 75° C. at a solvent:feed ratio of 5:1. Equilibrium data and calculated distribution coefficients for ethanol are shown in TABLE 2 below.

TABLE 2

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.1000 | 0.0396 | 0.0164 | 0.4622 | 0.997 |
| | 0.3000 | 0.2000 | 0.0923 | | |
| | 0.0500 | 0.0389 | 0.0158 | | |
| | 0.1500 | 0.1193 | 0.0567 | | |

Cetyl alcohol showed an excellent distribution coefficient for ethanol and no indication of the formation of emulsions. It was easily separated from the raffinate phase due to the large difference in densities.

### Example 4

Mixtures of Cetyl Alcohol and Other Solvents For Extraction of Ethanol. Mixtures of cetyl alcohol with other solvents were used in order to produce a resulting extraction solvent having a lower melting point than pure cetyl alcohol. For instance, a solvent mixture containing 50 wt % cetyl alcohol and 50 wt % oleic acid was found to have a melting point lower than 35° C. This solvent mixture was tested for ethanol extraction at a solvent:feed ratio of 5:1 at 75° C. Equilibrium data and calculated distribution coefficients for ethanol are shown in TABLE 3 below.

TABLE 3

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.1000 | 0.0395 | 0.0165 | 0.4313 | 0.998 |
| | 0.2000 | 0.0762 | 0.0316 | | |
| | 0.3000 | 0.1150 | 0.0486 | | |
| | 0.5000 | 0.1938 | 0.0848 | | |

The solvent mixture demonstrated an excellent distribution coefficient for the extraction of ethanol from a dilute aqueous mixture.

### Example 5

Mixtures of Cetyl Alcohol With Aromatic Solvents For Extraction. Mixtures of an aromatic solvent and cetyl alcohol were used in order to produce a resulting extraction solvent having a lower melting point. For instance, a solvent mixture containing 50 wt % cetyl alcohol and 50 wt % mixed xylenes was also found to have a melting point lower than 30° C. This solvent mixture was tested for extraction of ethanol and butanol from an ABE (acetone/butanol/ethanol) feed at a solvent:feed ratio of 5:1 at 75° C. and 40° C. Equilibrium data and calculated distribution coefficients for ethanol and butanol are shown in TABLES 4 and 5 below, respectively.

TABLE 4

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.03 | 0.0021 | 0.0009 | 0.3038 | 0.9906 |
| | 0.06 | 0.0043 | 0.0012 | | |
| | 0.12 | 0.0084 | 0.0025 | | |
| | 0.15 | 0.0105 | 0.0031 | | |
| | 0.19 | 0.0125 | 0.0039 | | |
| | 0.002 | 0.00007 | 0.00003 | | |
| 40 | 0.03 | 0.0025 | 0.0005 | 0.2097 | 0.9983 |
| | 0.06 | 0.0052 | 0.0010 | | |
| | 0.12 | 0.0102 | 0.0021 | | |
| | 0.15 | 0.0126 | 0.0027 | | |
| | 0.19 | 0.0152 | 0.0032 | | |
| | 0.002 | 0.00009 | 0.00002 | | |

TABLE 5

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.03 | 0.0002 | 0.0008 | 4.1040 | 0.9877 |
| | 0.06 | 0.0005 | 0.0017 | | |
| | 0.12 | 0.0010 | 0.0039 | | |
| | 0.16 | 0.0012 | 0.0049 | | |
| | 0.20 | 0.0014 | 0.0060 | | |
| | 0.060 | 0.00026 | 0.00129 | | |
| 40 | 0.03 | 0.0003 | 0.0008 | 3.3992 | 0.9888 |
| | 0.06 | 0.0006 | 0.0017 | | |
| | 0.12 | 0.0011 | 0.0037 | | |
| | 0.16 | 0.0014 | 0.0050 | | |
| | 0.20 | 0.0017 | 0.0058 | | |
| | 0.060 | 0.00038 | 0.00113 | | |

The solvent mixture demonstrated a high distribution coefficient for ethanol as shown in TABLE 4 and an even higher distribution coefficient for butanol as shown in TABLE 5.

### Example 6

Xylenes as an Extraction Solvent for Butanol. Mixed xylenes (a mixture of o-xylene, m-xylene and p-xylene), which is a common hydrocarbon solvent, were tested as an extraction solvent for a dilute solution of n-butanol in water at a solvent:feed ratio of 5:1 at 22° C. and 40° C., both of which are at or below normal fermentation temperatures. Equilibrium data and calculated distribution coefficients are shown in TABLE 6 below.

TABLE 6

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 40 | 0.01 | 0.0069 | 0.0075 | 1.1494 | 0.9836 |
| | 0.02 | 0.0037 | 0.0039 | | |
| | 0.04 | 0.0098 | 0.0117 | | |
| | 0.06 | 0.0018 | 0.0021 | | |
| 22 | 0.01 | 0.0054 | 0.0036 | 0.7195 | 0.9888 |
| | 0.02 | 0.0027 | 0.0017 | | |
| | 0.04 | 0.0103 | 0.0070 | | |
| | 0.06 | 0.0141 | 0.0106 | | |

As can be seen, the distribution coefficient for n-butanol was an order of magnitude greater than that of ethanol, indicating that xylenes are an excellent solvent for recovery of n-butanol from dilute aqueous feeds.

### Example 7

Mixed Xylenes as an Extraction Solvent for ABE Fermentation. Mixed xylenes were tested as an extraction solvent for a dilute solution of acetone, n-butanol and ethanol in water (products of ABE fermentation, wherein each component was present at less than 1 wt %, typical of many ABE fermentation broths) at a solvent:feed ratio of 5:1 at 40° C. and 75° C. Equilibrium data and calculated distribution coefficients for butanol are shown in TABLE 7 below.

TABLE 7

| Temperature | Feed | Raffinate | Extract | Distribution | |
|---|---|---|---|---|---|
| (° C.) | (Wₒ/W_{w}) | (Wₒ/W_{w}) | (Wₒ/Wₛ) | Coefficient | R² |
| 75 | 0.005 | 0.0005 | 0.0009 | 1.9257 | 0.9976 |
| | 0.01 | 0.0009 | 0.0018 | | |
| | 0.02 | 0.0019 | 0.0036 | | |
| | 0.025 | 0.0025 | 0.0047 | | |
| | 0.03 | 0.0030 | 0.0059 | | |
| 40 | 0.005 | 0.0018 | 0.0016 | 0.9677 | 0.964 |
| | 0.01 | 0.0009 | 0.0008 | | |
| | 0.02 | 0.0038 | 0.0031 | | |
| | 0.025 | 0.0043 | 0.0043 | | |
| | 0.03 | 0.0052 | 0.0054 | | |

### Example 8

Batch Separation of Ethanol from Xylenes Using Zeolite Molecular Sieves. To simulate an extract phase from liquid-liquid extraction, a feed stream was made up containing 8.00 wt % ethanol, 0.07 wt % water and the balance mixed xylenes. The result was a single phase, clear solution. 10.0 grams of this feed stream were placed into each of seven stoppered flasks to which were added 5.0 gm Linde 5 Å zeolite pellets (⅛″ extrudate). The zeolite had been previously activated by heating to 400° C. and then cooling in a dessicator. The flasks were placed in a hot air oven maintained at 40° C. and a timer was started. Samples of liquid were withdrawn as a function of time and analyzed by gas chromatography for ethanol. FIG. 6 shows an illustrative plot of ethanol content as a function of contact time with the 5 Å zeolite pellets. As shown in FIG. 6, the ethanol content leveled out at 1.07 wt %, thereby indicating that the capacity of the 5 Å zeolite pellets for ethanol (plus the very small amount of water present) was approximately 14 wt %, which is consistent with other observations. Comparable results were obtained when the flasks were evacuated to <10 torr using a vacuum pump (also see FIG. 6).

### Example 9

Column Separation of Ethanol from Xylenes Using Zeolite Molecular Sieves. Next a column adsorption experiment was run to simulate an industrial process. Commercially available Linde 5 Å molecular sieves containing binder were ground and sieved to 100-120 mesh using standard screens. After activation at 400° C., the sieves were cooled to 150° C. and poured directly into mixed xylenes. It should be noted that the color of the powder in xylenes was light brown.

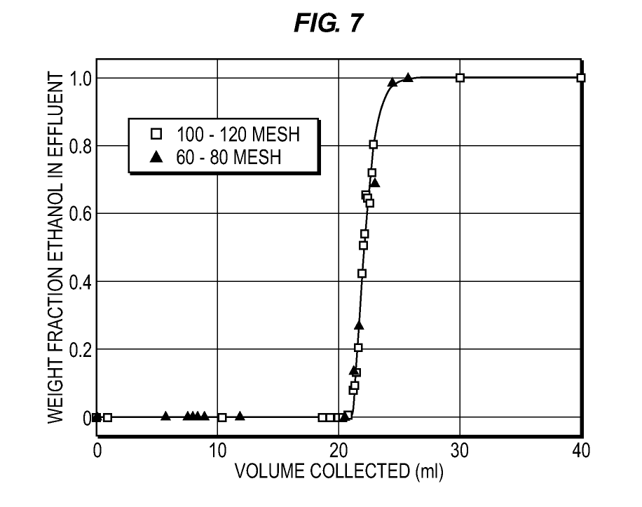

Approximately 6.0 grams of 5 Å sieves were loaded wet into a 10.1 mm ID glass chromatography column which contained a 200 ml reservoir at the top, an outlet stopcock to control flow and which had been fitted with cotton at the base to support the molecular sieves. After the powder had settled, the level of xylenes was lowered to the top of the solids after which 20.0 ml of a feed stream containing 7.47 wt % ethanol and 0.07 wt % water were carefully added to the top of the bed. The stopcock was opened and samples of liquid were taken every 10 minutes for analysis by gas chromatography. Data were normalized to produce a dimensionless concentration by dividing each of the outlet ethanol concentrations by that of the feed stream. FIG. 7 shows an illustrative plot of ethanol weight fraction versus volume of effluent collected. As can be seen in FIG. 7, the breakthrough curve was extremely sharp and no ethanol appeared in the product until 20 ml of effluent had exited the column. This result strongly supports the use of 5 Å sieves to recover ethanol from xylenes as described in the present disclosure. A similar experiment was run using 60-80 mesh 5 Å sieves, and as shown in FIG. 7, there was no apparent difference in the breakthrough curve.

Although the present disclosure has been described with reference to specific embodiments, these descriptions are not meant to be construed in a limiting sense. Various modifications of the disclosed embodiments, as well as alternative embodiments will become apparent to persons having ordinary skill in the art upon reference to the description. It will be understood that certain of the above-described structures, functions, and operations of the above-described embodiments are not necessary to practice such embodiments and are included in the description simply for completeness of an exemplary embodiment or embodiments. In addition, it will be understood that specific structures, functions, and operations set forth in the above and below described referenced patents and publications can be practiced in conjunction with various embodiments, but they are not essential. It is therefore to be understood that embodiments may be practiced otherwise than as specifically described without actually departing from the spirit and scope of the disclosure. From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

What is claimed is the following:

1. A method for extracting an organic solute from a feed source, said method comprising:
providing a feed source;
wherein the feed source comprises an organic solute;
transferring the feed source to a liquid-liquid extraction unit;
wherein the liquid-liquid extraction unit comprises a plurality of equilibrium stages;
contacting the feed source with an extraction solvent in each of the plurality of equilibrium stages;
wherein the extraction solvent comprises at least one aromatic solvent;
wherein the extraction solvent is substantially immiscible with the feed source; and
wherein contacting comprises transferring at least a portion of the organic solute from the feed source to the extraction solvent; and
recovering a raffinate phase substantially depleted of the organic solute and an extract phase substantially enriched in the organic solute;
wherein organic solute is dissolved in the extraction solvent in the extract phase.

2. The method of claim 1, wherein the extraction solvent further comprises a solvent modifier.

3. The method of claim 2, wherein the solvent modifier is selected from the group consisting of long chain alcohols, long chain fatty acids, esters of long chain fatty acids, naturally occurring oils, and combinations thereof.

4. The method of claim 3, wherein the solvent modifier is a solid at room temperature.

5. The method of claim 2, wherein the solvent modifier comprises cetyl alcohol.

6. The method of claim 1, further comprising:
removing any residual extraction solvent from the raffinate phase.

7. The method of claim 1, further comprising:
removing the organic solute from the extract phase; and
recycling the extraction solvent after removing the organic solute.

8. The method of claim 1, wherein the feed source is a fermentation feed source.

9. The method of claim 1, wherein the feed source comprises a biofuel.

10. The method of claim 1, wherein the feed source is substantially devoid of solids.

11. The method of claim 1, wherein the organic solute comprises at least one compound selected from the group consisting of acetone, butanol, isobutanol, ethanol and combinations thereof.

12. The method of claim 1, further comprising:
applying an electric field to the extraction solvent and the feed source while contacting occurs;
wherein the electric field increases a surface area of the extraction solvent dispersed in the feed source.

13. The method of claim 1, further comprising:
passing the extract phase through at least one bed of 3 Å molecular sieve zeolites; and
after passing the extract phase through the at least one bed of 3 Å molecular sieve zeolites, passing the extract phase through at least one bed of 5 Å molecular sieve zeolites;
wherein the 5 Å molecular sieve zeolites adsorb the organic solute.

14. The method of claim 13, further comprising:
recovering the organic solute from the 5 Å molecular sieve zeolites.

15. A method for extracting an organic solute from a feed source, said method comprising:
providing a feed source;
wherein the feed source comprises an organic solute;
wherein the organic solute comprises at least one compound selected from the group consisting of acetone, butanol, isobutanol, ethanol and combinations thereof;
transferring the feed source to a liquid-liquid extraction unit;
wherein the liquid-liquid extraction unit comprises a plurality of equilibrium stages;
contacting the feed source with an extraction solvent in each of the plurality of equilibrium stages;
wherein the extraction solvent comprises cetyl alcohol and at least one aromatic solvent;
wherein the extraction solvent is substantially immiscible with the feed source; and
wherein contacting comprises transferring at least a portion of the organic solute from the feed source to the extraction solvent; and
recovering a raffinate phase substantially depleted of the organic solute and an extract phase substantially enriched in the organic solute;
wherein the organic solute is dissolved in the extraction solvent in the extract phase.

16. The method of claim 15, further comprising:
passing the extract phase through at least one bed of 3 Å molecular sieve zeolites; and
after passing the extract phase through the at least one bed of 3 Å molecular sieve zeolites, passing the extract phase through at least one bed of 5 Å molecular sieve zeolites;
wherein the 5 Å molecular sieve zeolites adsorb the organic solute.

17. The method of claim 16, further comprising:
recovering the organic solute from the 5 Å molecular sieve zeolites.

18. The method of claim 15, further comprising:
passing the extract phase through at least one bed of 5 Å molecular sieve zeolites;
wherein the 5 Å molecular sieve zeolites adsorb the organic solute.

19. A method for extracting an organic solute from water, said method comprising:
providing an organic solute dissolved in water; and
extracting the organic solute from the water using an extraction solvent comprising cetyl alcohol.

20. The method of claim 19, wherein extracting takes place in a liquid-liquid extraction unit.

21. The method of claim 20, wherein the liquid-liquid extraction unit comprises a plurality of equilibrium stages.

22. The method of claim 19, wherein the organic solute is an alcohol.

23. A method for separating an organic solute from a feed source, said method comprising:
providing a feed source;
wherein the feed source comprises an organic solute dissolved in an extraction solvent;
passing the feed source through at least one bed of 3 Å molecular sieve zeolites;
wherein the 3 Å molecular sieve zeolites remove any residual water from the feed source and form a dewatered feed source; and
passing the dewatered feed source through at least one bed of 5 Å molecular sieve zeolites;
wherein the 5 Å molecular sieve zeolites adsorb the organic solute and form a substantially solute-free dewatered feed source;
wherein the substantially solute-free dewatered feed source comprises spent extraction solvent.

24. The method of claim 23, further comprising:
recovering the organic solute from the 5 Å molecular sieve zeolites.

25. The method of claim 24, wherein recovering comprises heating the 5 Å molecular sieve zeolites.

26. The method of claim 24, wherein recovering comprises applying a vacuum to the 5 Å molecular sieve zeolites.

27. The method of claim 24, wherein recovering comprises heating and applying a vacuum to the 5 Å molecular sieve zeolites.

28. The method of claim 23, further comprising:
recycling the spent extraction solvent.

29. The method of claim 23, wherein there are two beds of 3 Å molecular sieve zeolites and two beds of 5 Å molecular sieve zeolites.

30. The method of claim 29, wherein the two beds of 3 Å molecular sieve zeolites and the two beds of 5 Å molecular sieve zeolites are each operated in a swing bed fashion.

31. The method of claim 23, wherein the organic solute is an alcohol.

32. The method of claim 31, wherein the alcohol is ethanol.

33. The method of claim 31, wherein the alcohol is butanol.

34. The method of claim 23, wherein the organic solute has a molecular diameter of not more than about 4 Å.

35. The method of claim 23, wherein the extraction solvent comprises at least one aromatic solvent.

36. The method of claim 35, wherein the extraction solvent further comprises a solvent modifier.

37. The method of claim 36, wherein the solvent modifier comprises cetyl alcohol.

38. An apparatus comprising:
a solvent input line;
a solvent transfer line;
a first bed of 3 Å molecular sieve zeolites and a second bed of 3 Å molecular sieve zeolites linked to the solvent input line;
a first bed of 5 Å molecular sieve zeolites and a second bed of 5 Å molecular sieve zeolites linked to the first bed of 3 Å molecular sieve zeolites and the second bed of 3 Å molecular sieve zeolites by the solvent transfer line; and
an output line linked to the first bed of 5 Å molecular sieve zeolites and the second bed of 5 Å molecular sieve zeolites.

39. The apparatus of claim 38, wherein the first bed of 3 Å molecular sieve zeolites and the second bed of 3 Å molecular sieve zeolites are operable in a swing bed fashion.

40. The apparatus of claim 38, wherein the first bed of 5 Å molecular sieve zeolites and the second bed of 5 Å molecular sieve zeolites are operable in a swing bed fashion.

41. The apparatus of claim 38, further comprising:
a heater coupled to the first bed of 5 Å molecular sieve zeolites and the second bed of 5 Å molecular sieve zeolites.
